(19) United States
(12) Patent Application Publication
(10) Pub. No.: US 2008/0220421 A1
(43) Pub. Date: Sep. 11, 2008

(54) ISOTHERMAL SCREENING FOR NUCLEIC ACIDS ASSOCIATED WITH DISEASES AND CONDITIONS OF THE GI TRACT

(76) Inventor: Frank G. Salinas, Wheaton, IL (US)

Correspondence Address: MCANDREWS HELD & MALLOY, LTD, 500 WEST MADISON STREET, SUITE 3400, CHICAGO, IL, 60661, US

(21) Appl. No.: 11/679,699

(22) Filed: Feb. 27, 2007

Related U.S. Application Data
(60) Provisional application No. 60/777,169, filed on Feb. 27, 2006.

(51) Int. Cl. C12Q 1/68

(52) U.S. Cl. 435/6

(57) ABSTRACT

The presently described technology relates generally to the art of molecular diagnostics and more particularly to point-of-care diagnostic methods and materials. The diagnostic methods and materials of the presently described technology are suitable for a variety of uses including but not limited to the bedside or field diagnosis of infectious or noninfectious diseases.

### RELATED APPLICATIONS

The present application is related to and claims priority from U.S. Provisional Patent Application Ser. No. 60/777,169, filed Feb. 27, 2006, the contents of which are hereby incorporated herein by reference in their entirety. Additionally, all cited references in the present application are hereby incorporated by reference in their entirety.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The presently described technology relates generally to the art of molecular diagnostics and more particularly to point-of-care diagnostic methods and materials. The diagnostic methods and materials of the presently described technology are suitable for a variety of uses including but not limited to the bedside or field diagnosis of infectious or noninfectious diseases. In particular, the presently described technology relates to the methods and materials for the detecting, diagnosing, staging, monitoring, prognosticating, or determining the predisposition of an individual to diseases and conditions of the GI tract, such as GI tract cancer. For example, the presently described technology relates to the screening and detection of target nucleic acid sequences in a test sample related to diseases and conditions of the GI tract, such as GI tract cancer.

The organs of the GI tract include the esophagus, stomach, small and large intestines, rectum and pancreas. Of the approximately 225,900 new cases of GI tract cancer projected for the United States during 1996, 131,200 will be due to colorectal cancer. Further, GI tract cancers will account for approximately 127,070 related deaths (American Cancer Society statistics). In addition to its high incidence, GI tract cancers can be extremely lethal; for example, greater than 97% of pancreatic cancer patients will die of the disease. H. J. Wanebo, et al., Cancer 78:580-91 (1996).

Generally, the early detection of GI tract cancers at a pre-invasive stage dramatically reduces disease-related mortality. However, only few GI tract cancers are detected at this stage. For example, only 37% of colorectal cancers are detected at this stage by screening for premalignant polyps which can be removed before they progress to cancer. The primary methods used for colorectal cancer screening are fecal occult blood testing (FOBT) and flexible sigmoidoscopy. A. M. Cohen et al. In: Cancer: Principles and Practice of Oncology, Fourth Edition, pp. 929-977, Philadelphia, Pa.: J/B. Lippincott Co. (1993). Although FOBT is noninvasive, simple and inexpensive, its sensitivity is low; for example, sensitivity for detecting colorectal cancer was only 26% in one study. D. A. Ahlquist et al., JAMA 269: 1262-1267 (1993). Further, although flexible sigmoidoscopy is highly sensitive for detecting early cancer and precursor polyps, it is invasive, costly, and too technically demanding to be used for routine screening. D. F. Ransohoff, et al., JAMA 269: 1278-1281 (1993). In addition, only eight percent (8%) of pancreatic cancers and eighteen percent (18%) of stomach cancers are detected at a pre-invasive stage (American Cancer Society statistics). Thus, the need exists for improved screening methods for detection of GI tract diseases such as cancer.

### BRIEF SUMMARY OF THE INVENTION

One object of the present invention is to provide a molecular diagnostic system comprising methods and materials for the isothermal screening and detection of nucleic acids. Still another object of the present invention is to provide a molecular diagnostic system comprising methods and reagents for the isothermal screening and detection of nucleic acids associated with but not limited to disease, disease predisposition, disease causative agents, and any combination or derivative thereof. A further object of the present invention is to provide a molecular diagnostic system comprising methods and materials for the isothermal screening and detection of nucleic acids associated with diseases and conditions of the GI track.

One or more of the preceding objects, or one or more other objects which will become plain upon consideration of the present specification, are satisfied by the invention described herein.

One aspect of the invention, which satisfies one or more of the above objects, is a test kit having reagents for the isothermal detection of nucleic acids associated with but not limited to disease, disease predisposition, disease causative agents, and any combination or derivative thereof. Another aspect of the invention is a test kit comprising: a strand transferase component; a polymerase component; and one or more primers and/or probes complementary to one or more nucleic acids associated with but not limited to disease, disease predisposition, disease causative agents, and any combination or derivative thereof. One preferred aspect of the present invention is a test kit comprising: a reverse transcriptase, a strand transferase component; a DNA dependent DNA polymerase component; and one or more primers and/or probes complementary to one or more nucleic acids associated with diseases and conditions of the GI track.

### BRIEF DESCRIPTION OF SEVERAL VIEWS OF THE FIGURES

FIG. 1 is a schematic view of one aspect of the isothermal DNA amplification system of the present invention employing one primer complementary to a target nucleic acid, a strand transferase, and a polymerase.

FIG. 2 is a schematic view of another aspect of the isothermal DNA amplification system of the present invention employing two primers complementary to opposite strands and flanking a target nucleic acid, a strand transferase and a polymerase.

### DETAILED DESCRIPTION OF THE INVENTION

The present invention provides methods and materials for the isothermal screening and detection of nucleic acids associated with but not limited to disease, disease predisposition, disease causative agents, and any combination or derivative thereof. As used herein, and without limitation, nucleic acid generally includes any size DNA, RNA, DNA/RNA hybrid, or analog thereof. The nucleic acid can be single stranded, double stranded, or a combination of single and double stranded. As used herein, and without limitation, disease generally includes an impairment of the normal state of the living animal or plant body or one of its parts that interrupts or modifies the performance of the vital functions, is typically manifested by distinguishing signs and symptoms, and is a response to environmental factors (as malnutrition, industrial hazards, or climate), to specific infective agents (as parasites, bacteria, or viruses), to inherent defects of the organism (as genetic anomalies), or to combinations or derivatives of these factors.

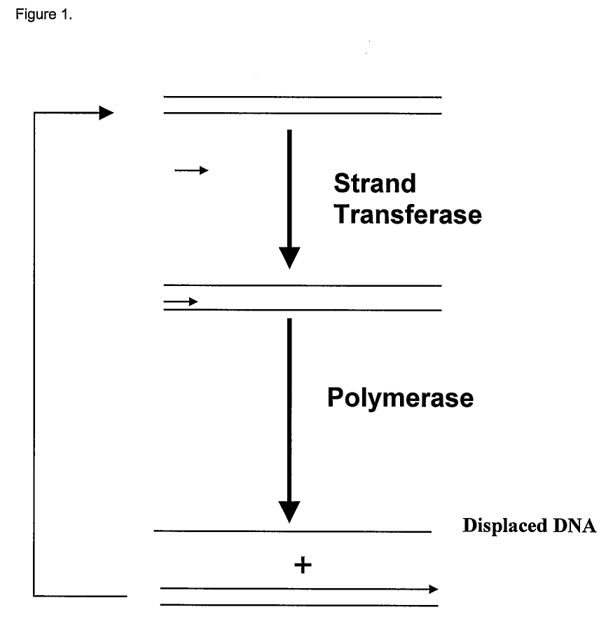

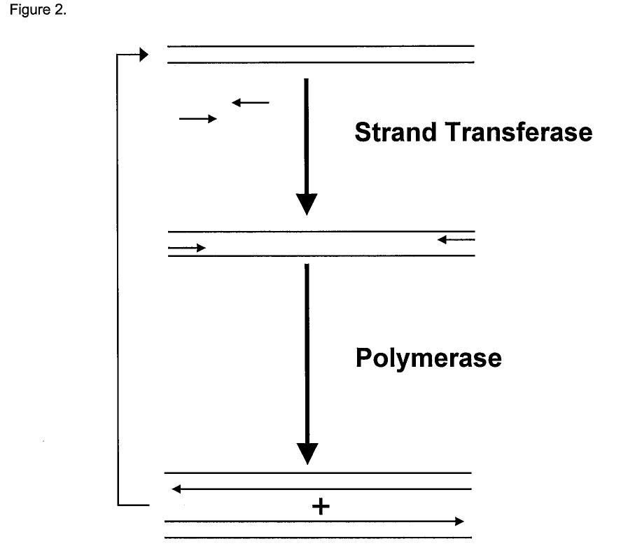

One aspect of the present invention includes methods and materials for the quantitative or qualitative isothermal screening and detection of one or more target nucleic acids of interest. This aspect of the present invention comprises contacting the target nucleic acid with at least one nucleic acid primer having complementarity to the target nucleic acid, a strand transferase, and a polymerase. The strand transferase catalyzes the homologous pairing of the at least one primer to a specific location on the target nucleic acid to form a primer-template junction that is acted upon by the polymerase to replicate and amplify the target nucleic acid (FIG. 1). In one preferred embodiment, the target nucleic acid is contacted with two primers complementary to opposite strands and flanking said target nucleic acid, in the presence of a strand transferase and a polymerase (FIG. 2). In certain aspects of the present invention, the isothermal amplification of the nucleic acid is performed as describe in U.S. Pat. No. 6,929,915, Methods for Nucleic Acid Manipulation. This reference is herein incorporated by reference.

As used herein without limitation, a strand transferase generally is a catalyst for the identification and base pairing of homologous sequences between nucleic acids, a process also known as homologous pairing or strand exchange. Bianco et al provides a general discussion of strand transferases in “DNA strand exchange proteins: a biochemical and physical comparison” at Front Biosci. 1998 Jun. 17; 3:D570-603. This reference is herein incorporated by reference. Strand transferases can be derived from either a prokaryotic system or an eukaryotic system, including but not limited to yeast, bacteria, and bacteriophages such as T4 and T7. For example West discusses eukaryotic strand transferases in Recombination genes and proteins” in Curr Opin Genet Dev. 1994 April; 4(2):221-8. This reference is herein incorporated by reference. Radding discussed the recA strand exchange protein in “Helical RecA nucleoprotein filaments mediate homologous pairing and strand exchange” at Biochim Biophys Acta. 1989 Jul. 7; 1008(2):131-45. This reference is herein incorporated by reference. Also, the UvsX strand transferase was described by Kodadek et al., The mechanism of homologous DNA strand exchange catalyzed by the bacteriophage T4 uvsX and gene 32 proteins” JBC 1988 Jul. 5; 263(19):9427-36. This reference is herein incorporated by reference. Yonesaki discusses T4 homologous recombination in “Recombination apparatus of T4 phage” at Adv Biophys. 1995; 31:3-22. This reference is herein incorporated by reference. Also, Salinas et. al have discussed the homology dependence of UvsX catalyzed strand exchange in “Homology dependence of UvsX protein-catalyzed joint molecule formation” at J Biol Chem. 1995 Mar. 10; 270(10):5181-6. This reference is herein incorporated by reference. Exemplar strand transferase proteins include but are not limited to the eukaryotic Rad51 protein, the bacterial recA protein, the bacterial phage T4 UvsX protein, the bacteriophage T7 gene 2.5 or any protein fragment, derivative, or homolog thereof, including proteins found in nature and those engineered or modified using recombinant DNA technology. Kong et. al has discussed T7 strand exchange in “Role of the bacteriophage T7 and T4 single-stranded DNA-binding proteins in the formation of joint molecules and DNA helicase-catalyzed polar branch migration.” J Biol Chem. 1997 Mar. 28; 272(13):8380-7. This reference is herein incorporated by reference.

Strand transferases generally operate by first binding single stranded regions of DNA to form a nucleoprotein filament generally referred to as the presynaptic filament. The presynaptic filament then binds a target nucleic acid and performs a search for homology that once complete results in the formation of a joint molecule or D-loop. Strand transferases generally have accessory protein factors that augment or modify their activity. For example, strand transferases generally have accessory protein factors that effect the formation and/or stability of the presynaptic filament under varying conditions, including for example buffer conditions and/or the presence of other proteins competing to bind regions of single-stranded nucleic acid. Exemplar strand transferase accessory proteins include but are not limited to the bacteriophage T4 UvsX accessory protein UvsY, the E. coli RecA accessory proteins RecFOR, the yeast and human Rad51 accessory protein Rad52, and any protein fragment, derivative, or homolog thereof, including proteins found in nature and those engineered or modified using recombinant DNA technology.

As used herein without limitation, a polymerase generally is any of several enzymes, such as DNA polymerase, RNA polymerase, or reverse transcriptase, that catalyze the formation of nucleic acid from precursor substances in the presence of preexisting nucleic acid acting as a template. The polymerase of the present invention can be derived from a eukaryotic or a prokaryotic system. For example the polymerase can be derived from a bacterium such as E. coli, a bacteriophage such as bacteriophage T4 or bacteriophage T7, a eukaryotic organism such as yeast or human, a virus, or any protein fragment, derivative, or homolog thereof, including proteins found in nature and those engineered or modified using recombinant DNA technology. Exemplar polymerases include but are not limited to the bacteriophage T4 gene product 43 protein, and any mutants or derivatives of the gene 43 protein including but not limited to the exonuclease deficient 43 exo⁻ polymerase. Benkovic et. al discusses replisome mediated DNA replication in “Replisome Mediated DNA Replication” at Annu Rev Biochem. 2001; 70:181-208. This reference is herein incorporated by reference.

Polymerases generally have accessory protein factors that augment or modify their activity. Exemplar polymerase accessory factors include but are not limited to clamp proteins and clamp loader proteins. Clamp proteins generally have affinity and/or a topological link to both the polymerase and the nucleic acid being acted upon by said polymerase, thereby forming a stable link between polymerase and nucleic acid, the result of which is the formation of a stable polymerase nucleic acid complex having high processivity Clamp loader proteins facilitate the assembly of a clamp protein onto a nucleic acid and can also facilitate and mediate a concomitant or subsequent interaction with the polymerase. As used herein in connection with certain aspects and embodiments of the invention, the term holoenzyme generally regards a polymerase-clamp complex.

Polymerase accessory factors can be derived from a bacterium such as E. coli, a bacteriophage such as bacteriophage T4 or bacteriophage T7, a eukaryotic organism such as yeast or human, a virus, or any protein fragment, derivative, or homolog thereof, including proteins found in nature and those engineered or modified using recombinant DNA technology. Exemplar clamp proteins include but are not limited to the bacteriophage T4 gene product 45 protein, and any mutants or derivatives of the T4 gene product 45 protein. Trakselis et discuss the T4 polymerase holoenzyme in Creating a dynamic picture of the sliding clamp during T4 DNA polymerase holoenzyme assembly by using fluorescence resonance energy transfer” at Proc Natl Acad Sci USA. 2001 Jul. 17; 98(15):8368-75. This reference is herein incorporated by reference.

In certain embodiments of the present invention, the quantitative or qualitative isothermal screening and detection of one or more target nucleic acids of interest is performed in the presence of a single stranded nucleic acid binding protein (SSB). SSB's used pursuant to the present invention can be derived from a bacterium such as E. coli, a bacteriophage such as bacteriophage T4 or bacteriophage T7, a eukaryotic organism such as yeast or human, or any protein fragment, derivative, or homolog thereof, including proteins found in nature and those engineered or modified using recombinant DNA technology. Exemplar SSB's include but are not limited to the E. coli SSB protein, the bacteriophage T4 gene product 32 protein, the bacteriophage T7 gene product 2.5 protein, and the yeast or human RPA protein, or any mutants or derivatives thereof.

In certain embodiments of the present invention, the quantitative or qualitative isothermal screening and detection of one or more target nucleic acids of interest is performed in the presence of a helicase, preferably a DNA helicase. The helicase can be derived from a prokaryote or a eukaryote. For example, the DNA helicase can be from a bacterium such as E. coli., a bacteriophage such as bacteriophage T4 or bacteriophage T7, a yeast, or human. Exemplar helicases include but are not limited to the bacteriophage T4 gene product 41, the bacteriophage T4 dda protein, the bacteriophage T7 gene 4 protein, the E. coli UvrD protein, and any mutants or derivatives thereof. For example, Salinas and Kodadek have discussed the role of DNA helicases during strand homologous recombination in “Phage T4 homologous strand exchange: a DNA helicase, not the strand transferase, drives polar branch migration.” Cell 1995 Jul. 14; 82(1):111-9. This reference is herein incorporated by reference. Also, Salinas and Benkovic have discussed the role of DNA helicases in bacteriophage T4 replication in “Characterization of bacteriophage T4-coordinated leading- and lagging-strand synthesis on a minicircle substrate.” Proc Natl Acad Sci USA. 2000 Jun. 20; 97(13):7196-201. This reference is herein incorporated by reference. Also, Alberts et al discusses the general nature of replication in bacteriophage T4 in “Studies on DNA replication in the bacteriophage T4 in vitro system” at Cold Spring Harb Symp Quant Biol. 1983; 47 Pt 2:655-68. This reference is herein incorporated by reference.

In certain other embodiments of the present invention, the quantitative or qualitative isothermal screening and detection of one or more target nucleic acids of interest is performed in the presence of a helicase and a helicase accessory factor. The DNA helicase and the DNA helicase accessory factor can be derived from a eukaryotic or prokaryotic system. For example, the DNA helicase and the DNA helicase accessory factor can be from a bacterial system such as E. coli. or a bacteriophage system such as bacteriophage T4. For example, one DNA helicase/accessory factor pair is the bacteriophage T4 gene product 41 protein and its accessory factor gene product 59 protein. Jones et al discusses the gene product 59 protein in “Bacteriophage T4 gene 41 helicase and gene 59 helicase-loading protein: a versatile couple with roles in replication and recombination” at Proc Natl Acad Sci USA. 2001 Jul. 17; 98(15):8312-8. This reference is herein incorporated by reference.

In still other embodiments of the present invention, the quantitative or qualitative isothermal screening and detection of one or more target nucleic acids of interest is performed in the presence of a primosome. As used herein a primosome is a term that generally characterizes a complex comprising a DNA helicase and an RNA polymerase usually referred to as a primase. The primosome is active in synthesizing RNA primers on the lagging strand of a replication fork for the initiation of Okazaki fragment synthesis during coordinated leading- and lagging strand synthesis. Primases can be derived from a prokaryote or a eukaryote. For example, the primase can be from a bacterium such as E. coli., a bacteriophage such as bacteriophage T4 or bacteriophage T7, a yeast, or a human. One exemplar primase is the bacteriophage T4 gene product 61 protein, and derivatives or mutants thereof.

The phrase “amplification reaction reagents” as used herein includes but is not limited to reagents which are well known for their use in nucleic acid amplification reactions and may include but are not limited to: a single or multiple reagent, reagents, enzyme or enzymes separately or individually having reverse transcriptase and/or polymerase activity, strand transferase activity, or exonuclease activity; enzyme cofactors such as magnesium or manganese; salts; nicotinamide adenine dinucleotide (NAD); and deoxynucleoside triphosphates (dNTPs) such as, for example, deoxyadenosine triphosphate, deoxyguanosine triphosphate, deoxycytodine triphosphate and thymidine triphosphate. Other reagents include molecular crowding agents, including but not limited to polyethylene glycol PEG 8000. The exact amplification reagents employed are largely a matter of choice for one skilled in the art based upon the particular amplification reaction employed. For example, it is known in the art that volume occuping agents, or molecular crowding agents, inhance the activity or function of strand transferases, polymerases, and their accessory factors. The following references are herein incorporated by reference: (1) “Enhancement of recA Protein-promoted DNA Strand Exchange Activity by Volume occupying agents” at J Biol Chem. 1992 May 5; 267(13):9307-14; (2) “Stimulation of the processivity of the DNA polymerase of bacteriophage T4 by the polymerase accessory proteins” at J Biol Chem. 1991 Jan. 25; 266(3):1830-40; (3) “Macromolecular crowding”: thermodynamic consequences for protein-protein interactions within the T4 DNA replication complex: The role of ATP hydrolysis”; (4) “Macromolecular crowding”: thermodynamic consequences for protein-protein interactions within the T4 DNA replication complex” at J Biol Chem. 1990 Sep. 5; 265(25):15160-7; (5) “Assembly of a functional replication complex without ATP hydrolysis: a direct interaction of bacteriophage T4 gp45 with T4 DNA polymerase” at Proc Natl Acad Sci USA. 1993 Apr. 15; 90(8):3211-5; and (6) “A coupled complex of T4 DNA replication helicase (gp41) and polymerase (gp43) can perform rapid and processive DNA strand-displacement synthesis” at Proc Natl Acad Sci USA. 1996 Dec. 10; 93(25):14456-61.

### Target Nucleic Acids

Target nucleic acids of the present invention include but are not limited to those nucleic acids associated with the development or onset of a disease state, including for example those nucleic acids that show the presence of specific infective agents or inherent defects of in an organism's genome. Target nucleic acids include but are not limited to either or both nucleic acids exogenous or endogenous to the organism being screened. Exemplar target nucleic acids belonging to specific infective agents of interest include but are not limited to those nucleic acids derived from protozoa, parasites, fingi, bacteria, viruses, and combinations or derivatives thereof.

The primer sets provided herein can be employed according to the isothermal DNA amplification disclosed herein. Probe sequences are also provided. The probe sequences can be combined with various primer sets to form oligonucleotide or “oligo” sets that can be used to amplify and detect a target sequence.

A set of contiguous and partially overlapping cDNA sequences designated as CS141 and transcribed from GI tract tissue, has previously been described in U.S. Pat. No. 6,867,016 entitled “Reagents and methods useful for detecting diseases of the gastrointestinal tract.” This reference is herein incorporated by reference. Also provided in U.S. Pat. No. 6,867,016 are sequences useful as primers and/or probes in combination with the strand transferase dependent isothermal DNA amplification system described herein and in U.S. Pat. No. 6,929,915, for the detecting, diagnosing, staging, monitoring, prognosticating, preventing or treating, or determining the predisposition of an individual to diseases and conditions of the GI tract, such as GI tract cancer.

One aspect of the present invention provides a method of detecting a target CS141 polynucleotide in a test sample which comprises contacting the test sample with a strand transferase, a polymerase, and at least one CS141-specific polynucleotide and detecting the presence of the target CS141 polynucleotide in the test sample. The CS141-specific polynucleotide has at least 50% identity with a polynucleotide selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, SEQUENCE ID NO 7, SEQUENCE ID NO 8, SEQUENCE ID NO 9, SEQUENCE ID NO 10, SEQUENCE ID NO 11, SEQUENCE ID NO 12, SEQUENCE ID NO 13 (“SEQUENCE ID NOS 1-13”), and fragments or complements thereof. Also, the CS141-specific polynucleotide may be attached to a solid phase prior to performing the method.

Another embodiment of the present invention also provides a method for detecting CS141 mRNA in a test sample, which comprises performing reverse transcription (RT) with at least one primer in order to produce cDNA, amplifying the cDNA so obtained using the strand transferase dependent isothermal DNA amplification system described herein, and CS141 oligonucleotides as sense and antisense primers to obtain CS141 amplicon, and detecting the presence of the CS141 amplicon as an indication of the presence of CS141 mRNA in the test sample, wherein the CS141 oligonucleotides have at least 50% identity to a sequence selected from the group consisting of SEQUENCE ID NOS 1-13, and fragments or complements thereof. Also, the test sample can be reacted with a solid phase prior to performing the method, prior to amplification or prior to detection. This reaction can be a direct or an indirect reaction. Further, the detection step can comprise utilizing a detectable label capable of generating a measurable signal. The detectable label can be attached to a solid phase.

Another embodiment of the present invention further provides a method of detecting a target CS141 polynucleotide in a test sample suspected of containing target CS141 polynucleotides, which comprises (a) contacting the test sample with at least one CS141 oligonucleotide as a sense primer and at least one CS141 oligonucleotide as an anti-sense primer, and amplifying same to obtain a first stage reaction product; (b) contacting the first stage reaction product with at least one other CS141 oligonucleotide to obtain a second stage reaction product, with the proviso that the other CS141 oligonucleotide is located 3′ to the CS141 oligonucleotides utilized in step (a) and is complementary to the first stage reaction product; and (c) detecting the second stage reaction product as an indication of the presence of a target CS141 polynucleotide in the test sample. The CS141 oligonucleotides selected as reagents in the method have at least 50% identity to a sequence selected from the group consisting of SEQUENCE ID NOS 1-13, and fragments or complements thereof. Amplification is performed by the isothermal strand transferase dependent amplification herein described. The test sample can be reacted either directly or indirectly with a solid phase prior to performing the method, or prior to amplification, or prior to detection. The detection step also comprises utilizing a detectable label capable of generating a measurable signal; further, the detectable label can be attached to a solid phase. Test kits useful for detecting target CS141 polynucleotides in a test sample are also provided which comprise a container containing at least one CS141-specific polynucleotide selected from the group consisting of SEQUENCE ID NOS 1-13, and fragments or complements thereof. These test kits further comprise containers with tools useful for collecting test samples (such as, for example, blood, urine, saliva and stool). Such tools include lancets and absorbent paper or cloth for collecting and stabilizing blood; swabs for collecting and stabilizing saliva; and cups for collecting and stabilizing urine or stool samples. Collection materials, such as papers, cloths, swabs, cups, and the like, may optionally be treated to avoid denaturation or irreversible adsorption of the sample. The collection materials also may be treated with or contain preservatives, stabilizers or antimicrobial agents to help maintain the integrity of the specimens. The test kits will also contain reagents and materials for the isothermal strand transferase dependent amplification system described herein, including for example a strand transferase, a polymerase, and strand transferase and/or polymerase accessory factors.

Another embodiment of the present invention also provides a purified polynucleotide or fragment thereof derived from a CS141 gene having utility in combination with the isothermal strand transferase dependent amplification system described herein. The purified polynucleotide is capable of selectively hybridizing to the nucleic acid of the CS141 gene, or a complement thereof. The polynucleotide has at least 50% identity with a sequence selected from the group consisting of (a) SEQUENCE ID NOS 1-9, SEQUENCE ID NO 12, SEQUENCE ID NO 13, and complements thereof, and (b) fragments of SEQUENCE ID NOS 1-9. Further, the purified polynucleotide can be produced by recombinant and/or synthetic techniques. The purified recombinant polynucleotide can be contained within a recombinant vector.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., PNA as defined hereinbelow) which can be used to identify a specific polynucleotide present in samples bearing the complementary sequence.

A polynucleotide “derived from” or “specific for” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The sequence may be complementary or identical to a sequence which is unique to a particular polynucleotide sequence as determined by techniques known in the art. Comparisons to sequences in databanks, for example, can be used as a method to determine the uniqueness of a designated sequence. Regions from which sequences may be derived, include but are not limited to, regions encoding specific epitopes, as well as non-translated and/or non-transcribed regions.

The derived polynucleotide will not necessarily be derived physically from the nucleotide sequence of interest under study, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide. In addition, combinations of regions corresponding to that of the designated sequence may be modified in ways known in the art to be consistent with the intended use.

A “fragment” of a specified polynucleotide refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the specified nucleotide sequence.

The term “polynucleotide” as used herein means a polymeric form of nucleotides of any length, either ribonucleotides or deoxyribonucleotides. This term refers only to the primary structure of the molecule. Thus, the term includes double- and single-stranded DNA, as well as double- and single-stranded RNA. It also includes modifications, such as methylation or capping and unmodified forms of the polynucleotide. The terms “polynucleotide,” “oligomer,” “oligonucleotide,” and “oligo” are used interchangeably herein.

“A sequence corresponding to a cDNA” means that the sequence contains a polynucleotide sequence that is identical or complementary to a sequence in the designated DNA. The degree (or “percent”) of identity or complementarity to the cDNA will be approximately 50% or greater, preferably at least about 70% or greater, and more preferably at least about 90% or greater. The sequence that corresponds to the identified cDNA will be at least about 50 nucleotides in length, preferably at least about 60 nucleotides in length, and more preferably at least about 70 nucleotides in length. The correspondence between the gene or gene fragment of interest and the cDNA can be determined by methods known in the art and include, for example, a direct comparison of the sequenced material with the cDNAs described, or hybridization and digestion with single strand nucleases, followed by size determination of the digested fragments.

“Purified polynucleotide” refers to a polynucleotide of interest or fragment thereof which is essentially free, e.g., contains less than about 50%, preferably less than about 70%, and more preferably less than about 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

Methods for amplifying and detecting a nucleic acid in a test sample generally comprise contacting a test sample with a strand transferase, a polymerase, amplification reagents and a previously mentioned primer set to form a reaction mixture. The reaction mixture is then placed under amplification conditions to form an amplification product to thereby amplify the target sequence. Amplification products may be detected using a variety of detection technologies. Preferably, however, an amplification product/probe hybrid is formed and detected as an indication of the presence of a target nucleic acid in the test sample.

The primer sets provided herein comprise two oligonucleotide primers that can be employed to amplify a target sequence in a test sample. The term “test sample” as used herein, means anything suspected of containing a target sequence of interest. The test sample is, or can be derived from, any biological source, such as for example, blood, seminal fluid, ocular lens fluid, cerebral spinal fluid, milk, ascites fluid, synovial fluid, peritoneal fluid, amniotic fluid, tissue, fermentation broths, cell cultures and the like. The test sample can be used (i) directly as obtained from the source or (ii) following a pre-treatment to modify the character of the sample. Thus, the test sample can be pre-treated prior to use by, for example, preparing plasma from blood, disrupting cells or viral particles, preparing liquids from solid materials, diluting viscous fluids, filtering liquids, distilling liquids, concentrating liquids, inactivating interfering components, adding reagents, purifying nucleic acids, and the like.

A “target sequence” as used herein includes but is not limited to a nucleic acid sequence that is amplified, detected, or both amplified and detected using the primer sets herein provided. Additionally, while the term target sequence is sometimes referred to as single stranded, those skilled in the art will recognize that the target sequence may actually be double stranded. Thus, in cases where the target is double stranded, primer sequences of the present invention will amplify both strands of the target sequence.

The primer sets that can be employed to amplify a target sequence preferably comprise deoxyribonucleic acid (DNA), or ribonucleic acid (RNA). Such primer sets can be employed according to isothermal DNA amplification disclosed herein. Additionally, in light of the RNA nature of mRNA, the isothermal nucleic acid amplification technology disclosed herein, and the primer sets may be employed in combination with a reverse transcriptase. Briefly, the reverse transcriptase provides a method of transcribing a strand of DNA from an RNA target sequence. The copied DNA strand transcribed from the RNA target is commonly referred to as “cDNA” which then can serve as a template for amplification by the isothermal nucleic acid amplification system mentioned above. The process of generating cDNA shares many of the hybridization and extension principles surrounding the isothermal nucleic acid amplification system described herein, but at least one of the enzymes employed should have reverse transcriptase activity. Enzymes having reverse transcriptase activity are well known and therefore don't warrant further discussion. Additionally, other methods for synthesizing cDNA are also known and include commonly owned U.S. patent application Ser. No. 08/356,287 filed Feb. 22, 1995, which is herein incorporated by reference. Generally, therefore, amplifying a target sequence in a test sample will generally comprise the steps of contacting a test sample with a primer set, a strand transferase, a polymerase, and amplification reagents to form a reaction mixture and placing the reaction mixture under amplification conditions to thereby amplify the target sequence.

Amplification products produced using the primer sets provided herein may be detected using a variety of detection technologies well known in the art. For example, amplification products may be detected using agarose gel electrophoresis and visualization by ethidium bromide staining and exposure to Ultraviolet (UV) light or by sequence analysis of the amplification product for confirmation of a target nucleic acid.

Alternatively, amplification products may be detected by oligonucleotide hybridization with a probe. Probe sequences generally are 10 to 50 nucleotides long, more typically 15 to 40 nucleotides long, and similarly to primer sequences, probe sequences are also nucleic acid. Hence, probes may comprise DNA, RNA or nucleic acid analogs such as uncharged nucleic acid analogs including but not limited to peptide nucleic acids (PNAs) which are disclosed in International Patent Application WO 92/20702 or morpholino analogs which are described in U.S. Pat. Nos. 5,185,444, 5,034,506, and 5,142,047 all of which are herein incorporated by reference. Such sequences can routinely be synthesized using a variety of techniques currently available. For example, a sequence of DNA can be synthesized using conventional nucleotide phosphoramidite chemistry and the instruments available from Applied Biosystems, Inc, (Foster City, Calif.); DuPont, (Wilmington, Del.); or Milligen, (Bedford, Mass.). Similarly, and when desirable, all nucleic acids disclosed herein, including but not limited to amplified target nucleic acids, primers, probes, or any combination thereof can be labeled using methodologies well known in the art such as described in U.S. Pat. Nos. 5,464,746; 5,424,414; and 4,948,882 all of which are herein incorporated by reference. Additionally, probes typically hybridize with the target sequence between the primer sequences. In other words, the probe sequence typically is not coextensive with either primer.

The term “label” as used herein means a molecule or moiety having a property or characteristic which is capable of detection. A label can be directly detectable, as with, for example, radioisotopes, fluorophores, chemiluminophores, enzymes, colloidal particles, fluorescent microparticles and the like; or a label may be indirectly detectable, as with, for example, specific binding members. It will be understood that directly detectable labels may require additional components such as, for example, substrates, triggering reagents, light, and the like to enable detection of the label. When indirectly detectable labels are used, they are typically used in combination with a “conjugate”. A conjugate is typically a specific binding member which has been attached or coupled to a directly detectable label. Coupling chemistries for synthesizing a conjugate are well known in the art and can include, for example, any chemical means and/or physical means that does not destroy the specific binding property of the specific binding member or the detectable property of the label. As used herein, “specific binding member” means a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

Probe sequences can be employed using a variety of homogeneous or heterogeneous methodologies to detect amplification products. Generally all such methods employ a step where the probe hybridizes to a strand of an amplification product to form an amplification product/probe hybrid. The hybrid can then be detected using labels on the amplified product, the primer, the probe or any combination thereof. Examples of homogeneous detection platforms for detecting amplification products include the use of FRET (fluorescence resonance energy transfer) labels attached to probes that emit a signal in the presence of the target sequence. So-called TaqMan assays described in U.S. Pat. No. 5,210,015 (herein incorporated by reference) and Molecular Beacon assays described in U.S. Pat. No. 5,925,517 (herein incorporated by reference) are examples of techniques that can be employed to homogeneously detect nucleic acid sequences. According to homogenous detection techniques, products of the amplification reaction can be detected as they are formed or in a so-called real time manner. As a result, amplification product/probe hybrids are formed and detected while the reaction mixture is under amplification conditions.

Heterogeneous detection formats typically employ a capture reagent to separate amplified sequences from other materials employed in the reaction. Capture reagents typically are a solid support material that is coated with one or more specific binding members specific for the same or different binding members. A “solid support material”, as used herein, refers to any material which is insoluble, or can be made insoluble by a subsequent reaction. Solid support materials thus can be a latex, plastic, derivatized plastic, magnetic or non-magnetic metal, glass or silicon surface or surfaces of test tubes, microtiter wells, sheets, beads, microparticles, chips, and other configurations known to those of ordinary skill in the art. To facilitate detection of an amplification product/probe hybrid in a heterogeneous type manner, primer or probes or both can be labeled with a first binding member which is specific for its binding partner which is attached to a solid support material such as a microparticle. Similarly, primers may be labeled with a second binding member specific for a conjugate as defined above. The amplification products bound to the probes can then be separated from the remaining reaction mixture by contacting the reaction mixture with the above solid support and then removing the solid support from the reaction mixture. Any amplification product/probe hybrids bound to the solid support may then be contacted with a conjugate to detect the presence of the hybrids on the solid support.

Whether detected in a homogeneous or heterogeneous manner, methods for detecting a target sequence in a test sample will generally comprise the steps of contacting a test sample with a primer set provided herein, a strand transferase, a polymerase, and amplification reagents to form a reaction mixture. The reaction mixture then is placed under amplification conditions to form an amplification product, as specified above. The amplification product is then detected as an indication of the presence of the target sequence in the test sample. As stated above, the reaction product may be detected using gel electrophoresis, heterogeneous methods or homogeneous methods. Accordingly, the reaction product may be detected in the reaction mixture while it is under amplification conditions with homogeneous techniques. Alternatively, the amplification product may be detected after amplification of the target sequence is complete using heterogeneous techniques or gels.

The present invention also provides oligonucleotide sets useful for amplifying and detecting a target nucleic acid sequence related to diseases and conditions of the GI tract in a test sample. These oligonucleotide sets, or “oligo sets”, comprise a primer set and a molecular beacon probe that can be used in the manner set forth above. Additionally, the oligo sets may be packaged in suitable containers and provided with additional reagents such as, for example, amplification reagents (also in suitable containers) to provide kits for amplifying and detecting a target nucleic acid sequence related to diseases and conditions of the GI tract in a test sample.

In one embodiment of the present invention, a test sample is reacted with a primer set either alone or in combination with other primer sets in a multiplex reaction. The test sample and the primers are reacted in combination with a strand transferase, a polymerase, and amplification reagents to produce an amplified target nucleic acid that can be detected either by detection of a labeled probe, or by detection of a labeled incorporated nucleotide, or by detection of a labeled primer, or by a combination thereof.

In another embodiment of the present invention, a test sample is reacted with a single primer, or with two or more single primers selected from different primer sets in a multiplex reaction, whereby said primer or primers are complementary to the same strand of nucleic acid. The test sample and the primers can be reacted in combination with a strand transferase, a polymerase, and amplification reagents to produce amplified single stranded target nucleic acid that can be detected either by the detection of a labeled probe, or by the detection of an labeled incorporated nucleotide, or by the detection of a labeled primer, or by a combination thereof.

In certain embodiments of the present invention, a target nucleic acid is detected by reacting a test sample and any one or more primers or primer sets with a reverse transcriptase polymerase to produce a first cDNA. Reaction of the test sample and the primer or primers with the reverse transcriptase to produce a cDNA is performed either before or concomitant with the admixture of a strand transferase, a polymerase, and amplification reagents. The amplified target nucleic acid can be detected either by the detection of a labeled probe, or by the detection of an labeled incorporated nucleotide, or by the detection of a labeled primer, or by a combination thereof.

While the invention has been described in detail and with reference to specific embodiments, it will be apparent to one skilled in the art that various changes and modifications may be made to such embodiments without departing from the spirit and scope of the invention.

SEQUENCE LISTINGS
SEQ ID NO: 1
GGNCAGAGCC TGCGCAGGGC AGGAGCAGCT GGCCCACTGG
CGGCCCGCAA CACTNCGTCT TNACCCTCTG GGCCCACTGC
ATCTAGAGGA GGGCCGTCTG TGAGGCCACT ACCCCTCCAG
CAACTGGGAG GTGGGACTGT CAGAAGCTGG CCCAGGGTGG
TGGTCAGCTG GGTCAGGGAC CTACGGCANC TGCTGGACCA
NCTNGNCTTT TCCATCGAAG CAGGGAAGTG GGAGCCTTGA
GCCCTTGGGT GGAAGCTTGA CCCCAAGCCA CTT
SEQ ID NO: 2
AGAGCCTGCG CAGGGCAGGA GCAGCTGGCC CACTGGCGGC
CCGCAACACT CCGTCTCACC CTCTGGGCNC ACTGCATCTA
GAGGAGGGCC GTCTGTNAGG CCACTACCCC TCCAGCAACT
GGGAGGTGGG ACTGTCAGAN GCTGGCCCAG GGTGGTGGTC
AGCTGGGTCA GGGACCTACG GCACCTGCTG GACCACCTCG
CCTTCTCCAT CGAAGCAGGG AANTGGGAGC CTCGAGCCCT
CGGGTGGAAG
SEQ ID NO: 3
TGGCGGCCCG CAACACTCCG TCTCACCCTC TGGGCCCACT
GCATCTAGAG GAGGGCCGTC TGTGAGGNCA CTACCCCTCC
AGCAACTGGG AGGTGGGACT GTCAGAATCT GGCCCAGGGT
GGTGGTCAGC TGGGTCAGGG ACCTACGGCA CCTGCTGGAC
CACCTCGCCT TCTCCATCGA AGCAGGGAAG TGGGAGCCTC
GAGCCCTCGG GTGGAAGCTG ACCCCAAGCC ANNCTTCACC
TGGACAGGAT
SEQ ID NO: 4
CCCTCTGGGC CCACTGCATC TAGAGGAGGG CCGTCTGTGA
GGCCACTACC CCTCCAGCAA CTGGGAGGTG GGACTGTCAG
AAGCTGGCCC AGGGTGGTGG TCAGCTGGGT CAGGGACCTA
TGGACCACCT CGCCTTCTCC ATCGAAGCAG GGAAGTGGGA
GCCTCGAGCC AGCTGACCCC AAGCCACCCT TCACCTGGAC
AGGATGAGAG TGT
SEQ ID NO: 5
CACGAGGGCC GTCTGTNAGG CCACTACCCC TCCAGCAACT
GGGAGGTGGG ACTGTCAGAN GCTGGCCCAG GGTGGTGGTC
AGCTGGGTCA GGGACCTACG GCACCTGCTG GACCACCTCG
CCTTCTCCAT CGAAGCAGGG AAGTGGGAGC CTCGAGCCCT
CGGGTGGAAG CTGACCCCAA GCCACCCTTC ACNTGGACAG
GATGAGAGTG TCAGGTGTGC TTCGCCTCCT GGCCCTCATC
TTTGCCATAG TCACGACATG GATGTTTATT CGAAGCTACA
TGAGCTT
SEQ ID NO: 6
GATGTTTATT CGAAGCTACA TGAGCTTCAG CATGAAAACC
ATCCGTCTGC CACGCTGGCT GGCCTCGCCC ACCAAGGAGA
TCCAGGTTAA AAAGTACAAG TGTGGCCTCA TCAAGCCCTG
CCCAGCCAAC TACTTTGCGT TTAAAATCTG CAGTGGGGCC
GCCAACGTCG TGGGCCCTAC TATGTGCTTT GAAGACCGCA
TGATCATGAG TCCTGTGAAA AACAATGTGG GCAGAGGCCT
AAACATCGCC CTGGTGAATG GAA
SEQ ID NO: 7
GTGAAAAACA ATGTGGGCAG AGGCCTAAAC ATCGCCCTGG
TGAATGGAAC CACGGGAGCT GTGCTGGGAC AGAAGGCATT
TGACATGTAC TCTGGAGATG TTATGCACCT AGTGAAATTC
CTTAAAGAAA TTCCGGGGGG TGCACTGGTG CTGGTGGCCT
CCTACGACGA TCCAGGGACC AAAATGAACG ATGAAAGCAG
GAAACTCTTC TCTGACTTGG GGAGTTCC
SEQ ID NO: 8
GGGGGGTGCA CTGGTGCTGG TGGCCTCCTA CGACGATCCA
GGGACCAAAA TGAACGATGA AAGCAGGAAA CTCTTCTCTG
ACTTGGGGAG TTCCTACGCA AAACAACTGG GCTTCCGGGA
CAGCTGGGTC TTCATAGGAG CCAAAGACCT CAGGGGTAAA
AGCCCCTTTG AGCAGTTCTT CCAGACACAA ACAAATACGA
GGGATGGCCA GAGCTGCTGG AGATGGAGGG CTGCATGCCC C
SEQ ID NO: 9
GGGATGGCCA GAGCTGCTGG AGATGGAGGG CTGCATGCCC
CCGAAGCCAT TTTAGGGTGG CTGTGGCTCT TCCTCAGCCA
GGGGCCTGAA GAAGCTCCTG CCTGACTTAG GAGTCAGAGC
CCGGCAGGGG CTGAGGAGGA GGAGCAGNGG GTGCTGCGTG
GAAGGTGCTG CAAGTCCTTG AAAGNNG
SEQ ID NO: 10
TTTTTTTTTT TCAAAACCAG CAAAAATAAA ATTTAATTGG
GCTCAAGTCT GGGCAGTTTG TCCTTCCTCA GGACCAGCCG
TCAGCAGTCC CTGACGAAAG CACCCCATTC TCTCCACAGA
CAGCTGGTTC CAGAAGGACC CTCTGAGGCT GGTCTTCCGG
GTAGGATGTG CTGTGGGAGG GTTCTGTTTC CGAGGAGGAG
AGGCGCGACA CAGCGTGCAA GGACCTGCAG CACCTTCCAC
GCAGCACCCC CTGCTCCTCC TCCTCAGCCC CTGCCGGGCT
CTGACTCCTA AGTCAGGCAG G
SEQ ID NO: 11
TTTTTCAAAA CCAGCAAAAA TAAAATTTAA TTGGGCTCAA
GTCTGGGCAG TTTGTCCTTC CTCAGGACCA GCCGTCAGCA
GTCCCTGACG AAAGCACCCC ATTCTCTCCA CAGACAGCTG GTT
SEQ ID NO: 12
GATGTTTATT CGAAGCTACA TGAGCTTCAG CATGAAAACC
ATCCGTCTGC CACGCTGGCT GGCCTCGCCC ACCAAGGAGA
TCCAGGTTAA AAAGTACAAG TGTGGCCTCA TCAAGCCCTG
CCCAGCCAAC TACTTTGCGT TTAAAATCTG CAGTGGGGCC
GCCAACGTCG TGGGCCCTAC GAAGACCGCA TGATCATGAG
TCCTGTGAAA AACAATGTGG GCAGAGGCCT AAACATCGCC
CTGGTGAATG GAACCACGGG AGCTGTGCTG GGACAGAAGG
CATTTGACAT GATGTTATGC ACCTAGTGAA ATTCCTTAAA
GAAATTCCGG GGGGTGCACT GCCTCCTACG ACGATCCAGG
GACCAAAATG AACGATGAAA GCAGGAAACT CTTCTCTGAC
TTGGGGAGTT CCTACGCAAA ACAACTGGGC TTCCGGGACA
GCTGGGTCTT CATAGGAGCC AAAGACCTCA GGGGTAAAAG
CCCCTTTGAG CAGTTCTTAA AGAACAGCCC AGACACAAAC
AAATACGAGG GATGGCCAGA GCTGCTGGAG ATGGAGGGCT
GCATGCCCCC TAGGGTGGCT GTGGCTCTTC CTCAGCCAGG
GGCCTGAAGA AGCTCCTGCC TGACTTAGGA GTCAGAGCCC
GGCAGGGGCT GAGGAGGAGG AGCAGGGGGT GCTGCGTGGA
AGGTGCTGCA GGTCCTTGCA CGCTGTGTCG CGCCTCTCCT
CCTCGGAAAC AGAACCCTCC CACAGCACAT CCTACCCGGA
AGACCAGCCT CAGAGGGTCC TTCTGGAACC AGCTGTCTGT
GGAGAGAATG GGGTGCTTTC GTCAGGGACT GCTGACGGCT
GGTCCTGAGG AAGGACAAAC TGCCCAGACT TGAGCCCAAT
TAAATTTTAT TTTTGCTGGT AAAAAMAAAW AAMMA
SEQ ID NO: 13
GGNCAGAGCC TGCGCAGGGC AGGAGCAGCT GGCCCACTGG
CGGCCCGCAA CACTCCGTCT CACCCTCTGG GCCCACTGCA
TCTAGAGGAG GGCCGTCTGT GAGGCCACTA CCCCTCCAGC
AACTGGGAGG TGGGACTGTC AGAAGCTGGC CCAGGGTGGT
GGTCAGCTGG GTCAGGGACC TACGGCACCT GCTGGACCAC
CTCGCCTTCT CCATCGAAGC AGGGAAGTGG GAGCCTCGAG
CCCTCGGGTG GAAGCTGACC CCAAGCCACC CTTCACCTGG
ACAGGATGAG AGTGTCAGGT GTGCTTCGCC TCCTGGCCCT
CATCTTTGCC ATAGTCACGA CATGGATGTT TATTCGAAGC
TACATGAGCT TCAGCATGAA AACCATCCGT CTGCCACGCT
GGCTGGCCTC GCCCACCAAG GAGATCCAGG TTAAAAAGTA
CAAGTGTGGC CTCATCAAGC CCTGCCCAGC CAACTACTTT
GCGTTTAAAA TCTGCAGTGG GGCCGCCAAC GTCGTGGGCC
CTACTATGTG CTTTGAAGAC CGCATGATCA TGAGTCCTGT
GAAAAACAAT GTGGGCAGAG GCCTAAACAT CGCCCTGGTG
AATGGAACCA CGGGAGCTGT GCTGGGACAG AAGGCATTTG
ACATGTACTC TGGAGATGTT TGAAATTCCT TAAAGAAATT
CCGGGGGGTG CACTGGTGCT GGTGGCCTCC TACGACGATC
CAGGGACCAA AATGAACGAT GAAAGCAGGA AACTCTTCTC
TGACTTGGGG AGTTCCTACG CAAAACAACT GGGCTTCCGG
GACAGCTGGG TCTTCATAGG AGCCAAAGAC CTCAGGGGTA
AAAGCCCCTT TGAGCAGTTC TTAAAGAACA GCCCAGACAC
AAACAAATAC GAGGGATGGC CAGAGCTGCT GGAGATGGAG
GGCTGCATGC CCCCGAAGCC ATTTTAGGGT GGCTGTGGCT
CTTCCTCAGC CAGGGGCCTG AAGAAGCTCC TGCCTGACTT
AGGAGTCAG GCCCGGCAGG GGCTGAGGAG GAGGAGCAGG
GGGTGCTGCG TGGAAGGTGC TGCAGGTCC TGCACGCTGT
GTCGCGCCTC TCCTCCTCGG AAACAGAACC CTCCCACAGC
ACATCCTAC CGGAAGACCA GCCTCAGAGG GTCCTTCTGG
AACCAGCTGT CTGTGGAGAG AATGGGGTG TTTCGTCAGG
GACTGCTGAC GGCTGGTCCT GAGGAAGGAC AAACTGCCCA
GACTTGAGC TTATTTTTGC TGGTTTTGAA AAAAAAAAA

What is claimed is:

1. A method for amplifying and detecting in a test sample a target nucleic acid sequence related to diseases and conditions of the GI tract, said method comprising contacting a test sample with a reverse transcriptase polymerase, a strand transferase, a DNA dependent DNA polymerase, and a primer having complementarity to nucleic acid associated with diseases and conditions of the GI track.

2. The method of claim 1 wherein said strand transferase is derived from a prokaryotic.

3. The method of claim 1 wherein said strand transferase is the uvsX strand transferase derived from the bacteriophage T4.

4. The polymerase of claim 1 wherein said DNA dependent DNA polymerase is derived from a prokaryotic.

5. The polymerase of claim 1 wherein said DNA dependent DNA polymerase is the gp43 polymerase derived from the bacteriophage T4.


SEQUENCE LISTING (13 sequences)

SEQ ID NO: 1 - DNA, 273 bp, Homo Sapiens
  misc_feature (3)..(3): base_polymorphism - note=  N′ represents an A or G or T or C polymorphism at this position
ggncagagcctgcgcagggcaggagcagctggcccactggcggcccgcaacactncgtcttnaccctctgggcccactgcatctagaggagggccgtctgtgaggccactacccctccagcaactgggaggtgggactgtcagaagctggcccagggtggtggtcagctgggtcagggacctacggcanctgctggaccanctngncttttccatcgaagcagggaagtgggagccttgagcccttgggtggaagcttgaccccaagccactt

SEQ ID NO: 2 - DNA, 250 bp, Homo Sapiens
  misc_feature (69)..(69): base_polymorphism - note=  N′ represents an A or G or T or C polymorphism at this position
agagcctgcgcagggcaggagcagctggcccactggcggcccgcaacactccgtctcaccctctgggcncactgcatctagaggagggccgtctgtnaggccactacccctccagcaactgggaggtgggactgtcagangctggcccagggtggtggtcagctgggtcagggacctacggcacctgctggaccacctcgccttctccatcgaagcagggaantgggagcctcgagccctcgggtggaag

SEQ ID NO: 3 - DNA, 250 bp, Homo Sapiens
  misc_feature (68)..(68): base_polymorphism - note=  N′ represents an A or G or T or C polymorphism at this position
tggcggcccgcaacactccgtctcaccctctgggcccactgcatctagaggagggccgtctgtgaggncactacccctccagcaactgggaggtgggactgtcagaatctggcccagggtggtggtcagctgggtcagggacctacggcacctgctggaccacctcgccttctccatcgaagcagggaagtgggagcctcgagccctcgggtggaagctgaccccaagccanncttcacctggacaggat

SEQ ID NO: 4 - DNA, 213 bp, Homo Sapiens
ccctctgggcccactgcatctagaggagggccgtctgtgaggccactacccctccagcaactgggaggtgggactgtcagaagctggcccagggtggtggtcagctgggtcagggacctatggaccacctcgccttctccatcgaagcagggaagtgggagcctcgagccagctgaccccaagccacccttcacctggacaggatgagagtgt

SEQ ID NO: 5 - DNA, 287 bp, Homo Sapiens
  misc_feature (17)..(17): base_polymorphism - note=  N′ represents an A or G or T or C polymorphism at this position
cacgagggccgtctgtnaggccactacccctccagcaactgggaggtgggactgtcagangctggcccagggtggtggtcagctgggtcagggacctacggcacctgctggaccacctcgccttctccatcgaagcagggaagtgggagcctcgagccctcgggtggaagctgaccccaagccacccttcacntggacaggatgagagtgtcaggtgtgcttcgcctcctggccctcatctttgccatagtcacgacatggatgtttattcgaagctacatgagctt

SEQ ID NO: 6 - DNA, 263 bp, Homo Sapiens
gatgtttattcgaagctacatgagcttcagcatgaaaaccatccgtctgccacgctggctggcctcgcccaccaaggagatccaggttaaaaagtacaagtgtggcctcatcaagccctgcccagccaactactttgcgtttaaaatctgcagtggggccgccaacgtcgtgggccctactatgtgctttgaagaccgcatgatcatgagtcctgtgaaaaacaatgtgggcagaggcctaaacatcgccctggtgaatggaa

SEQ ID NO: 7 - DNA, 228 bp, Homo Sapiens
gtgaaaaacaatgtgggcagaggcctaaacatcgccctggtgaatggaaccacgggagctgtgctgggacagaaggcatttgacatgtactctggagatgttatgcacctagtgaaattccttaaagaaattccggggggtgcactggtgctggtggcctcctacgacgatccagggaccaaaatgaacgatgaaagcaggaaactcttctctgacttggggagttcc

SEQ ID NO: 8 - DNA, 241 bp, Homo Sapiens
ggggggtgcactggtgctggtggcctcctacgacgatccagggaccaaaatgaacgatgaaagcaggaaactcttctctgacttggggagttcctacgcaaaacaactgggcttccgggacagctgggtcttcataggagccaaagacctcaggggtaaaagcccctttgagcagttcttccagacacaaacaaatacgagggatggccagagctgctggagatggagggctgcatgcccc

SEQ ID NO: 9 - DNA, 187 bp, Homo Sapiens
  misc_feature (148)..(148): base_polymorphism - note=  N′ represents an A or G or T or C polymorphism at this position
gggatggccagagctgctggagatggagggctgcatgcccccgaagccattttagggtggctgtggctcttcctcagccaggggcctgaagaagctcctgcctgacttaggagtcagagcccggcaggggctgaggaggaggagcagngggtgctgcgtggaaggtgctgcaagtccttgaaagnng

SEQ ID NO: 10 - DNA, 301 bp, Homo Sapiens
tttttttttttcaaaaccagcaaaaataaaatttaattgggctcaagtctgggcagtttgtccttcctcaggaccagccgtcagcagtccctgacgaaagcaccccattctctccacagacagctggttccagaaggaccctctgaggctggtcttccgggtaggatgtgctgtgggagggttctgtttccgaggaggagaggcgcgacacagcgtgcaaggacctgcagcaccttccacgcagcaccccctgctcctcctcctcagcccctgccgggctctgactcctaagtcaggcagg

SEQ ID NO: 11 - DNA, 123 bp, Homo Sapiens
tttttcaaaaccagcaaaaataaaatttaattgggctcaagtctgggcagtttgtccttcctcaggaccagccgtcagcagtccctgacgaaagcaccccattctctccacagacagctggtt

SEQ ID NO: 12 - DNA, 915 bp, Homo Sapiens
  misc_feature (946)..(946): base_polymorphism - note=  M′ represents an A or C at this position
gatgtttattcgaagctacatgagcttcagcatgaaaaccatccgtctgccacgctggctggcctcgcccaccaaggagatccaggttaaaaagtacaagtgtggcctcatcaagccctgcccagccaactactttgcgtttaaaatctgcagtggggccgccaacgtcgtgggccctacgaagaccgcatgatcatgagtcctgtgaaaaacaatgtgggcagaggcctaaacatcgccctggtgaatggaaccacgggagctgtgctgggacagaaggcatttgacatgatgttatgcacctagtgaaattccttaaagaaattccggggggtgcactgcctcctacgacgatccagggaccaaaatgaacgatgaaagcaggaaactcttctctgacttggggagttcctacgcaaaacaactgggcttccgggacagctgggtcttcataggagccaaagacctcaggggtaaaagcccctttgagcagttcttaaagaacagcccagacacaaacaaatacgagggatggccagagctgctggagatggagggctgcatgccccctagggtggctgtggctcttcctcagccaggggcctgaagaagctcctgcctgacttaggagtcagagcccggcaggggctgaggaggaggagcagggggtgctgcgtggaaggtgctgcaggtccttgcacgctgtgtcgcgcctctcctcctcggaaacagaaccctcccacagcacatcctacccggaagaccagcctcagagggtccttctggaaccagctgtctgtggagagaatggggtgctttcgtcagggactgctgacggctggtcctgaggaaggacaaactgcccagacttgagcccaattaaattttatttttgctggtaaaaamaaawaamma

SEQ ID NO: 13 - DNA, 1274 bp, Homo Sapiens
  misc_feature (3)..(3): base_polymorphism - note=  N′ represents an A or G or T or C polymorphism at this position
ggncagagcctgcgcagggcaggagcagctggcccactggcggcccgcaacactccgtctcaccctctgggcccactgcatctagaggagggccgtctgtgaggccactacccctccagcaactgggaggtgggactgtcagaagctggcccagggtggtggtcagctgggtcagggacctacggcacctgctggaccacctcgccttctccatcgaagcagggaagtgggagcctcgagccctcgggtggaagctgaccccaagccacccttcacctggacaggatgagagtgtcaggtgtgcttcgcctcctggccctcatctttgccatagtcacgacatggatgtttattcgaagctacatgagcttcagcatgaaaaccatccgtctgccacgctggctggcctcgcccaccaaggagatccaggttaaaaagtacaagtgtggcctcatcaagccctgcccagccaactactttgcgtttaaaatctgcagtggggccgccaacgtcgtgggccctactatgtgctttgaagaccgcatgatcatgagtcctgtgaaaaacaatgtgggcagaggcctaaacatcgccctggtgaatggaaccacgggagctgtgctgggacagaaggcatttgacatgtactctggagatgtttgaaattccttaaagaaattccggggggtgcactggtgctggtggcctcctacgacgatccagggaccaaaatgaacgatgaaagcaggaaactcttctctgacttggggagttcctacgcaaaacaactgggcttccgggacagctgggtcttcataggagccaaagacctcaggggtaaaagcccctttgagcagttcttaaagaacagcccagacacaaacaaatacgagggatggccagagctgctggagatggagggctgcatgcccccgaagccattttagggtggctgtggctcttcctcagccaggggcctgaagaagctcctgcctgacttaggagtcaggcccggcaggggctgaggaggaggagcagggggtgctgcgtggaaggtgctgcaggtcctgcacgctgtgtcgcgcctctcctcctcggaaacagaaccctcccacagcacatcctaccggaagaccagcctcagagggtccttctggaaccagctgtctgtggagagaatggggtgtttcgtcagggactgctgacggctggtcctgaggaaggacaaactgcccagacttgagcttatttttgctggttttgaaaaaaaaaaa